(19) United States
(12) Patent Application Publication
(10) Pub. No.: US 2025/0049090 A1
(43) Pub. Date: Feb. 13, 2025

(54) A NUTRITIONAL SUPPLEMENT FOR IMPROVED GUT HEALTH

(71) Applicant: Haleon US Holdings LLC, Wilmington, DE (US)

(72) Inventors: Simone PYLE, Whiteknights, Reading (GB); Morten Georg JENSEN, Delta Park (DK); Meet DESAI, Richmond, VA (US)

(21) Appl. No.: 18/719,308

(22) PCT Filed: Nov. 22, 2022
(86) PCT No.: PCT/US2022/080326

§ 371 Date: Jun. 13, 2024

Related U.S. Application Data
(60) Provisional application No. 63/291,621, filed on Dec. 20, 2021.

(51) Int. Cl. A23L 33/165; A23L 33/21; A61K 31/555; A61K 31/715; A61P 1/00

(52) U.S. Cl. CPC A23L 33/165; A23L 33/21; A61K 31/555; A61K 31/715; A61P 1/00

(57) ABSTRACT

Embodiments of the present invention are directed to a nutritional supplement composition comprising a dietary fiber and zinc carnosine.

### CROSS-REFERENCE TO RELATED APPLICATIONS

The present application is related to and claims priority from U.S. Provisional Patent Application 63/291,621 filed Dec. 20, 2021, which is incorporated herein by reference in its entirety.

### FIELD OF THE INVENTION

The present invention relates to nutritional supplement compositions. More particularly, the present invention relates to nutritional supplement compositions including at least a dietary fiber and zinc carnosine.

### BACKGROUND

The human gut is a critical component of digestion, breaking down complex carbohydrates, proteins, and fats in the lower gastrointestinal tract. Further, the gut microbiome contains a complex ecosystem of microorganisms that maintain homeostasis of the gastrointestinal tract and maintain human health.

A disruption of the fragile host-microbiota interaction equilibrium can play a role in the onset of several diseases. These diseases represent a growing worldwide health concern due to their increasing prevalence. For example, gut microbiota may contribute to the development of inflammatory diseases or disorders such irritable bowel syndrome (IBS).

Diet is an important factor influencing the composition of the gut microbiota. Typical modern diets may not provide sufficient nutrients to support a healthy gut metabolism. There may be nutritional products available on the market that are designed to supplement the nutrition that may be missing from a typical diet, but they are often ineffective or only include one or a limited number of active ingredients forcing the user to take multiple supplements to gain optimal benefits. Therefore, nutritional supplements for gut health that include multiple ingredients that work together to improve gut health are highly desirable.

### SUMMARY

The present invention is directed towards nutritional supplement compositions comprising a dietary fiber and zinc carnosine.

Particularly, nutritional supplement compositions of the invention comprise from about 700 mg to about 15.000 mg of the dietary fiber per serving, for example from about 700 mg to about 7400 mg, from about 700 mg to about 1500 mg or about 2600 mg to about 7400 mg. In certain embodiments the nutritional supplement compositions of the invention comprise about 1100 mg of dietary fiber per serving, about 3000 mg of dietary fiber per serving, about 3200 mg of dietary fiber per serving, about 4000 mg of dietary fiber per serving or about 10,000 mg of dietary fiber per serving.

Particularly, the nutritional supplement compositions of the invention comprise from about 10 mg to about 500 mg of zinc carnosine per serving. In certain embodiments the nutritional supplement compositions of the invention comprise from about 37 mg to about 500 mg of zinc carnosine per serving, from about 37 mg to about 150 mg of zinc carnosine per serving or about 37 mg to about 75 mg of zinc carnosine per serving. In certain embodiments the nutritional supplement compositions of the invention comprise from about 50 mg to about 500 mg of zinc carnosine per serving, from about 50 mg to about 150 mg of zinc carnosine per serving or from about 50 mg to about 75 mg of zinc carnosine per serving.

Particularly, the nutritional supplement compositions of the invention comprise a dietary fiber selected from the group consisting of wheat dextrin, hydrolysed guar gum and inulin.

In some embodiments, the dietary fiber is wheat dextrin. In some embodiments, the dietary fiber has a degree of polymerisation (DP) of 12-25. In some embodiments, the dietary fiber is wheat dextrin with a degree of polymerisation (DP) of 12-25.

The nutritional supplement compositions of the invention may further comprise one or more polyvalent metals, vitamins, minerals, enzymes, microorganisms, or a combination thereof.

The nutritional supplement compositions of the invention may be used to enhance gut health in a subject. Particularly, the nutritional supplement compositions of the invention may be used to enhance gut health in a subject by increasing the number of bacteria of Bifidobacterium spp in the gastrointestinal tract or feces of the subject. More particularly, the number of bacteria of Bifidobacterium spp. in the gastrointestinal tract or feces of the subject is increased in comparison to the number of bacteria of Bifidobacterium spp. in the gastrointestinal tract or feces of the subject prior to administration of the nutritional supplement composition.

There is also provided a method of improving gut metabolism in a subject comprising administering a nutritional supplement composition of the invention to said subject.

An embodiment of the present invention is directed to a nutritional supplement composition comprising a dietary fiber and zinc carnosine.

Another embodiment of the present invention is directed to a nutritional supplement composition comprising wheat dextrin as the dietary fiber. In an embodiment, the amount of wheat dextrin is between about 30% w/w and about 99% w/w based on the total weight of the composition.

Another embodiment of the composition is directed to a nutritional supplement composition comprising between about 10 mg and about 500 mg of zinc carnosine.

Another embodiment of the composition is directed to a nutritional supplement composition comprising wheat dextrin, zinc carnosine, and one or more polyvalent metals, vitamins, minerals, enzymes, microorganisms, or a combination thereof.

Another embodiment of the composition is directed a method of improving gut metabolism comprising administering the nutritional supplement composition.

### FIGURES

FIGS. 1A-1G illustrate bacterial enumeration (log 10 cells/mL) from in vitro batch culture after fermenting for 0, 8, 24, 48 and 72 h;

FIGS. 2A-2E illustrate bacterial enumeration (log 10 cells/mL) from in vitro batch culture after fermenting for 0, 8, 24, 48 and 72 h; and

FIG. 3 illustrates organic acid production (mM) from in vitro batch culture fermentation.

### DETAILED DESCRIPTION

Nutritional supplements of the present invention contain at least a dietary fiber and zinc carnosine (ZnC). Nutritional supplements are dietary supplements and may be used to complement daily food intake and improve gut metabolism, gut health, brain health, depression, and support overall wellbeing. The inventors have recognized that, surprisingly, the combination of dietary fiber and zinc carnosine in a single composition provides a synergistic effect and improves gut metabolism better than either one individually.

Chemical compounds typically referred to as vitamins and minerals provide significant value for maintaining an individual in a healthy state and/or for treating specific medical conditions. The human body cannot synthesize all the vitamins and minerals that are essential to maintaining the health of the human body. Thus, vitamins and minerals may be obtained from an external source. The two most common external sources are food and nutritional supplements. As most people do not eat foods that consistently provide the necessary daily requirements of vitamins and minerals, nutritional supplements are a valuable addition to a healthy diet.

Nutritional supplements of the present invention include at least one dietary fiber. Dietary fibers are complex carbohydrate polymers found in plants, which are not digested by the human digestive system. It is believed that the consumption of dietary fiber positively impacts the composition of gut microbiota. For example, dietary fiber balances bacteria in the gut, which is necessary to maintain health and prevent certain diseases or disorders.

Examples of dietary fibers that may be included in compositions of the present invention include, but are not limited to, inulin, fructooligosaccharides, galactooligosaccharides, arabinogalactan, maltodextrine, hydrolyzed guar gum, partially hydrolysed guar gum, wheat dextrin, beta-glucans, and combinations thereof. In some embodiments, the dietary fiber has a degree of polymerisation (DP) of 12-25. In some embodiments, the dietary fiber is not tapioca dextrin. The amount of dietary fiber included in compositions of the present invention may be any amount that imparts a therapeutic effect and is safe for human consumption. In an embodiment, the amount of dietary fiber may be between about 30% w/w and about 99% w/w based on the total weight of the composition.

In a preferred embodiment, the dietary fiber is wheat dextrin. In an embodiment, the composition contains between about 30% w/w and about 99% w/w of wheat dextrin based on the total weight of the composition. In another embodiment, the composition contains between about 50% w/w and about 70% w/w of wheat dextrin based on the total weight of the composition. In another embodiment, the composition contains between about 55% w/w and about 65% w/w of wheat dextrin based on the total weight of the composition.

In another embodiment, the dietary fiber is partially hydrolysed guar gum. In an embodiment, the composition contains between about 30% w/w and about 99% w/w of partially hydrolyzed guar gum based on the total weight of the composition. In another embodiment, the composition contains between about 50% w/w and about 70% w/w of partially hydrolyzed guar gum based on the total weight of the composition. In another embodiment, the composition contains between about 55% w/w and about 65% w/w of partially hydrolyzed guar gum based on the total weight of the composition.

The amount of dietary fiber may also be provided based on the weight of dietary fiber per serving. The term “per serving” refers to the amount of the composition that is intended to be administered, for example consumed, by a subject in a single sitting. In some embodiments the nutritional supplement compositions of the invention comprise from about 700 mg to about 15.000 mg of the dietary fiber per serving, for example from about 700 mg to about 7400 mg, from about 700 mg to about 1500 mg or about 2600 mg to about 7400 mg. In certain embodiments the nutritional supplement compositions of the invention comprise about 1100 mg of dietary fiber per serving, about 3000 mg of dietary fiber per serving, about 3200 mg of dietary fiber per serving, about 4000 mg of dietary fiber per serving or about 10,000 mg of dietary fiber per serving.

In some embodiments, the dietary fiber is wheat dextrin. In an embodiment, the composition contains between about 700 mg and about 7400 mg of wheat dextrin, such as between about 2600 mg and about 7400 mg of wheat dextrin. In certain embodiments, the composition comprises about 1100 mg of wheat dextrin. In certain embodiments, the composition comprises about 4000 mg of wheat dextrin. Particularly, the dietary fiber is wheat dextrin with a degree of polymerisation (DP) of 12-25.

In an embodiment, the dietary fiber is water-soluble and does not significantly change the viscosity and taste of the composition to which it is added or of an edible article to which the composition is added. In another embodiment, the dietary fiber is water insoluble.

Compositions of the present invention include ZnC. ZnC is an artificially produced derivative of carnosine, wherein zinc and carnosine are linked in a one-to-one ratio to provide a polymeric structure. L-carnosine has been shown to be a good carrier for zinc. For the avoidance of doubt, zinc carnosine or ZnC refers to the chelate compound.

Regulation of zinc is important to maintain overall health. Zinc has many functions one of which is to help maintain intestinal barrier integrity; alteration in gut barrier function allows permeation of detrimental microorganisms, antigens and proinflammatory factors. Zinc is believed to provide benefits in restoring the gastric lining, healing other parts of the gastrointestinal tract, improving taste disorders, improving GI disorders, and enhancing skin and liver. The primary mechanisms of action are thought to be related to its anti-inflammatory and antioxidant functions.

L-carnosine is a dipeptide composed of beta-alanine and L-histidine. It is naturally present in muscle and nerve cells and is thought to have antioxidant properties. The combination or chelation of the zinc and carnosine that results in ZnC is believed to have superior health benefits compared to either alone as carnosine enhances the absorption of zinc.

The amount of ZnC included in compositions of the present invention may be any amount that imparts a therapeutic effect and is safe for human consumption. In an embodiment, the amount of ZnC is between about 10 mg and about 500 mg per serving. In another embodiment, the amount of ZnC is between about 50 mg and 150 mg per serving. In another embodiment, the amount of ZnC is between about 37 mg and about 75 mg per serving.

In some embodiments, the nutritional supplement compositions of the invention comprise from about 10 mg to about 500 mg of zinc carnosine per serving. In certain embodiments the nutritional supplement compositions of the invention comprise from about 37 mg to about 500 mg of zinc carnosine per serving, from about 37 mg to about 150 mg of zinc carnosine per serving or about 37 mg to about 75 mg of zinc carnosine per serving. In certain embodiments the nutritional supplement compositions of the invention comprise from about 50 mg to about 500 mg of zinc carnosine per serving, from about 50 mg to about 150 mg of zinc carnosine per serving or from about 50 mg to about 75 mg of zinc carnosine per serving.

In a preferred embodiment, the composition includes at least wheat dextrin and ZnC. The inventors have recognized that, surprisingly, wheat dextrin combined with ZnC provides greater alteration in bacterial metabolism as compared to either individually or other available combinations. In particular, the combination of wheat dextrin and ZnC increases the presence of at least Bifidobacterium spp., Lactobacillus spp., Clostridium histolyticum group, Clostridium coccoides-Eubacterium rectale group, Clostridial cluster IX, acetate, butyrate and propionate. More particularly, the combination of wheat dextrin and ZnC increases the presence of at least Bifidobacterium spp., Lactobacillus spp., Clostridium histolyticum group, Clostridium coccoides-Eubacterium rectale group. Clostridial cluster IX, acetate, butyrate and propionate in the gastrointestinal tract or feces of a subject. Further, the combination of wheat dextrin and ZnC prolonged the increase of Bifidobacterium spp. These observed changes increase the effectiveness of the compositions of the present invention by imparting greater benefits to the gut and overall health of the user. For example, Bifidobacterium spp., and Lactobacillus spp. are considered health-positive bacteria and reduce acute diarrhea, improve immunity, decrease inflammation and related diseases, among other benefits.

Thus, nutritional supplement compositions of the invention may be used to enhance gut health in a subject. Particularly, the nutritional supplement compositions of the invention may be used to enhance gut health in a subject by increasing the number of bacteria of Bifidobacterium spp. in the gastrointestinal tract or feces of the subject. More particularly, the number of bacteria of Bifidobacterium spp. in the gastrointestinal tract or feces of the subject is increased in comparison to the number of bacteria of Bifidobacterium spp. in the gastrointestinal tract or feces of the subject prior to administration of the nutritional supplement composition.

Compositions according to the present invention may further include one or more polyvalent metals, vitamins, minerals, botanicals, enzymes, microorganisms, or a combination thereof.

The polyvalent metal may be iron, magnesium, zinc, selenium, copper, cobalt, manganese, molybdenum, vanadium, nickel, tin, chromium, or a combination thereof.

The vitamin may be vitamin C, vitamin E, vitamin A, vitamin A precursors, vitamin B6, vitamin D3, vitamin K, folic Acid, thiamin (Vitamin B1), riboflavin (vitamin B2), niacin (vitamin B3), pyridoxine (vitamin B6), cobalamins (Vitamin B12), pantothenic acid (vitamin B5), biotin, other B complex vitamins, choline, inositol, carotinoids, such as lutein, lycopene, zeaxanthin, and astaxanthin, or a combination thereof.

The mineral may be, iron, iodine, magnesium zinc, selenium, copper, calcium, manganese, silicon, molybdenum, vanadium, boron, nickel, tin, phosphorus, chromium, cobalt, chloride, potassium, any salts thereof, or a combination thereof.

The enzymes may be amylase, protease, bacterial protease, lipase, or a combination thereof. The microorganisms may be Bifidobacterium lactis or Lactobacillus acidophilus, or a combination thereof.

Compositions according to the present invention may comprise excipients and/or processing aides including, but not limited to, absorbents, diluents, flavorants, colorants, stabilizers, fillers, binders, disintegrants, lubricants, wetting agents, glidants, antiadherents, sugar or film coating agents preservatives, buffers, artificial sweeteners, natural sweeteners, dispersants, thickeners, solubilizing agents, and the like, or a combination thereof.

In an embodiment, the composition may be in any suitable dosage form. Exemplary dosage forms include, but are not limited to, tablets, caplets, capsules, chewable dosage forms, powder, sachet, gummies, liquids, and the like. The daily dosage may be included in a single delivery unit or may comprise multiple delivery units. In an embodiment, the dosage form may be solid. The solid dosage form may or may not include liquid or semi-solid components. In another embodiment, the dosage form is a gummy. In another embodiment, the dosage form is a powder.

In an embodiment. The composition may be consumed on its own. In another embodiment, the composition may be added to an edible article, which may be consumed. In an embodiment, the edible article may have a high moisture content or is a liquid. If the composition is added to an edible article that has a high moisture content or is a liquid, the composition quickly dissolves and blends into the contents of the product without imparting appreciable organoleptic properties and taste profile changes.

In an embodiment, compositions of the present invention improve gut health, gut microbiome, gastrointestinal barrier function, immune system function, relieve symptoms of IBS and other related diseases and/or diseases.

In one embodiment, there is provided a nutritional supplement composition that comprises, consists or consists essentially of about 1 mg Vitamin A, about 80.0 mg ascorbic acid, about 9.0 mg Vitamin E, about 11.0 mg zinc carnosine, about 10.0 mg Bacillus Coagulans (minimum 1 billion CFU), about 400.0 mg organic botanical blend (Inulin (Prebiotic Blue Agave Fiber), Astragalus Root Extract, Echinacea (aerial) Powder) and between about 2,600 mg and about 7.400 mg wheat dextrin.

In another embodiment, there is provided a nutritional supplement composition that comprises, consists, or consists essentially of about 4 mg Bifidobacterium Lactis, about 5 mg Lactobacillus acidophilus, about 50 mg of an enzyme blend (Amylase 2,000 DU, Protease 6,000 HUT, Bacterial Protease 5,000 PC/HUT, and Lipase 600 FIP), about 0.02 mg Vitamin D3, about 90 mg ascorbic acid, about 5 mg zinc oxide, about 50 mg elderberry (7% anthocyanins), about 4,000 mg wheat dextrin, about 100 mg of zinc carnosine, and an amount that is necessary of microcrystalline cellulose, filler (Dicalcium Phosphate. Anhydrous), silicon dioxide, and magnesium.

In another embodiment, there is provided a nutritional supplement composition that comprises, consists, or consists essentially of between about 4,000 mg and about 7,400 wheat dextrin, between about 75 mg and about 500 mg zinc carnosine, and an amount that is necessary of flavors and colors.

### EXAMPLES

### Examples 1-10—Examples of Compositions

Embodiments of the present invention may be prepared as indicated below in Examples 1-10.

### Example 1

| Ingredient | Amount (mg) |
|---|---|
| Vitamin A | 1.0 |
| Vitamin C (as ascorbic acid) | 80.0 |
| Vitamin E | 9.0 |
| Zinc Carnosine | 11.0 |
| Bacillus Coagulans (minimum 1 billion | 10.0 |
| CFU) | |
| Organic Botanical Blend (Inulin (Prebiotic | 400.0 |
| Blue Agave Fiber), Astragalus Root | |
| Extract, Echinacea (aerial) Powder) | |
| Wheat Dextrin | 2,600.0-7,400.0 |

### Example 2

| Ingredient | Amount (mg) |
|---|---|
| Bifidobacterium Lactis | 4.0 |
| Lactobacillus acidophilus | 5.0 |
| Enzyme Blend | 50.0 |
| Amylase 2,000 DU | |
| Protease 6,000 HUT | |
| Bacterial Protease 5,000 PC/HUT | |
| Lipase 600 FIP | |
| Vitamin D3 [Cholecaliferol 100 IU/mg] | 0.02 |
| Ascorbic Acid | 90.0 |
| Zinc oxide | 5.0 |
| Elderberry (7% anthocyanins) | 50.0 |
| Wheat dextrin | 2,600.0-7,400.0 |
| | (preferably, 4,000.0) |
| Zinc carnosine | 75.0-500.0 |
| | (preferably, 100.0) |
| Microcrystalline Cellulose | q.s. |
| Filler (Dicalcium Phosphate, Anhydrous) | q.s. |
| Silicon Dioxide | q.s. |
| Magnesium | q.s. |

### Example 3

| Ingredient | Amount (mg) |
|---|---|
| Wheat Dextrin | 2,600.0-7,400.0 (preferably, 4,000.0) |
| Zinc Carnosine | 75.0-500.0 |
| Flavors | q.s. |
| Colors | q.s. |

### Example 4

| Ingredient | Amount (mg) |
|---|---|
| Inulin | 2,600.0-7,400.0 (preferably, 3,200.0) |
| Zinc Carnosine | 75.0-500.0 |
| Flavors | q.s. |
| Colors | q.s. |

### Example 5

| Ingredient | Amount (mg) |
|---|---|
| Partially Hydrolyzed Guar Gum | 2,600.0-15,000.0 |
| | (preferably, 10,000.0) |
| Zinc Carnosine | 75.0-500.0 |
| Flavors | q.s. |
| Colors | q.s. |

### Example 6

| Ingredient | Amount (mg) |
|---|---|
| Wheat Dextrin | 7,400.0 |
| Zinc Carnosine | 75.0-500.0 |
| Flavors | q.s. |
| Colors | q.s. |

### Example 7

| Ingredient | Amount (mg) |
|---|---|
| Wheat Dextrin | 700.0-1,500.0 (preferably, 1,100.0) |
| Zinc Carnosine | 75.0-500.0 |
| Starch | 200.0-500.0 (preferably, 350.0) |
| Sorbitol | 600.0-1,100.0 (preferably, 850.0) |
| Dextrates | 70.0-150.0 (preferably, 100.0) |
| Silicified MCC | 100.0-200.0 (preferably, 150.0) |
| Anhydrous Citric Acid (Optional) | q.s. |
| Flavors | q.s. |
| Colors | q.s. |
| Aspartame | 1.0-5.0 |
| Magnesium Stearate | 12.0-40.0 |

### Example 8

| Ingredients | Amount (mg) |
|---|---|
| Wheat dextrin | 700.0-1,500.0 (preferably, 1,100.0) |
| Zinc Carnosine | 75.0-500.0 |
| Starch | 200.0-500.0 (preferably, 350.0) |
| Confectioner's sugar | 200.0-800.0 (preferably, 400.0) |
| Anhydrous Citric Acid | q.s. |
| Magnesium Stearate | 12.0-40.0 |
| Flavors | q.s. |
| Colors | q.s. |

### Example 9

| Ingredient | Amount (mg) |
|---|---|
| Wheat dextrin | 700.0-1,500.0 (preferably, 1,100.0) |
| Zinc Carnosine | 75.0-500.0 (preferably, 100.0) |
| Microcrystalline Cellulose | 150.0-300.0 (preferably, 200.0) |
| Starch | 20.0-100.0 (preferably, 50.0) |
| Magnesium Stearate | 5.0-20.0 (preferably, 10.0) |

### Example 10

| Ingredient | Amount (mg) |
|---|---|
| Inulin | 2,000.0-4,000.0 (preferably, 3,000.0) |
| Zinc carnosine | 75.0-500.0 (preferably, 100.0) |
| Sugar | 100.0-400.0 (preferably, 250.0) |
| Pectin | 40.0-80.0 (preferably 60.0) |
| Citric acid/sodium citrate | 15.0-45.0 (preferably, 25.0) |
| Flavor | q.s. |
| Color | q.s. |

### Example 11—Study—In Vitro Gut Model

### Summary

The present study assesses the impact of ZnC on gut microbial metabolism. Specifically, the aim of this study is to assess whether ZnC combined with dietary fibre (wheat dextrin (WD) and partially hydrolysed guar gum (PHGG)) alter bacterial metabolism compared to dietary fibre alone and/or ZnC alone.

In vitro gut model batch culture fermentations were run for 72 hours. Samples were collected for bacterial enumeration and organic acid production through fluorescent in situ hybridisation combined with flow cytometry and gas chromatography, respectively. Overall, the main findings were that the ZnC supplementation combined with WD increased in Bifidobacterium spp., and Clostridium histolyticum group but this finding was not shown in the vessels consisting of the ZnC components only. The vessels containing WD showed a greater alteration in bacterial metabolism compared to the PHGG vessels. Optimising the dose and substrate availability is important to ensure homeostasis and prevent potential pathogenic bacterial growth. ZnC may be an effective treatment for gastrointestinal diseases and/or disorders by contributing to intestinal barrier function, gut-brain axis and modulating the gut microbiota but dosing and substrate availability is important to ensure homeostasis and prevent pathogenic bacterial growth.

### Materials and Methods

### Subjects

Three healthy volunteers (28±4 years old) donated faecal samples. The volunteers had no history of gastrointestinal disorders, had not consumed antibiotics in the previous 3 months or prebiotic/probiotic supplements in the prior 2 weeks.

### Basal Media

Autoclaved basal medium (Peptone water 2 g, yeast extract 2 g, NaCl 0.1 g, K₂HPO₄ 0.04 g, KH₂PO₄ 0.04 g, MgSO₄·7H₂0 0.01 g, CaCl₂·6H₂0 0.01 g. NaHCO₃ 2 g. Tween 80 2 ml, haemin 0.05 g, vitamin K 10 μl, L-cysteine HCL 0.5 g and bile salt 0.5 g per litre) (Sigma, St. Louis, MO) was added to each vessel (135 ml per vessel) and incubated overnight in anaerobic conditions (oxygen-free nitrogen sparged at a rate of 15 ml/min).

### Faecal Sample

In the morning, participants brought a fresh faecal sample (<3 hours) to the laboratory in anaerobic jars (<1% O₂ and 9-13% CO₂) (AnaeroJar™ 2.5 L and AnaeroGem™, Thermo Fisher Scientific Oxoid Ltd, Basingstoke, Hampshire, UK). Samples were diluted with phosphate buffer saline (PBS) 10% (w/v) (pH 7.4) and homogenised (Stomacher 400, Seward, West Sussex, UK) for 2 minutes at 240 paddle beats per minute. Each vessel was inoculated with 15 ml of faecal slurry (total working volume of 300 ml).

### Substrates

In the first fermentation run, vessels comprised of V1 blank. V2 WD (3.6 g), V3 WD (3.6 g) and ZnC (45 mg), V4 WD (3.6 g) and L-carnosine (35.1 mg), V5 ZnC (45 mg), V6 WD (3.6 g) and zinc sulphate (9.9 mg) and V7 inulin (3.6 g) and ZnC (45 mg). The second batch cultures consisted of V1 blank. V2 PHGG (3.6 g), V3 PHGG (3.6 g) and ZnC (45 mg), V4 PHGG (3.6 g) and L-carnosine (35.1 mg), V5 ZnC (45 mg), V6 PHGG (3.6 g) and zinc sulphate (9.9 mg) and V7 inulin (3.6 g) and ZnC (45 mg).

Wheat dextrin (Benefiber, GSK, Warren, New Jersey, USA) is a resistant dextrin with a DP between 12-25 (Noack et al., 2013). Partially hydrolysed guar gum (Resource Optifiber. Nestlé Health Science, London, UK) comprises of galactose (α-1-6 bonds) and mannose units (β-1-4 bonds) with a molecular weight of 1,000 to 100.000 Da (average 20,000 Da) (Noack et al., 2013, Yoon et al., 2008). Inulin has an average DP of 12 and is made up of fructose joined by β-(2-1) linkages (Roberfroid, 2005). Zinc carnosine has the tradename PepZin GI (Hamari Chemicals Ltd, Japan). L-carnosine (L-carnosine 98%, ACROS Organic) and zinc sulphate (Zinc sulphide 99.99% (trace metal basis) ACROS organic) were purchased from Fisher Scientific (Fisher Scientific, Leicestershire, UK).

### Vessel Conditions

A water bath was used to ensure vessels remained at body temperature (37° C.) and a PH meter-controlled pH between 6.7-6.9. Vessels were continuously stirred throughout the experiment.

### Samples

A 750 μl sample was collected from each vessel at timepoints 0, 8, 24, 48 and 72 hours. The samples were centrifuged at 11,337×g for 5 minutes. The subsequent pellet was used for bacterial enumeration through fluorescence in situ hybridisation flow-cytometry (FISH-FCM) and 500 μl of supernatant used to assess organic acid production via gas chromatography. Bacterial probes used can be found in Table 1 below.

TABLE 1
Bacterial probes names and DNA sequence used to detect common gut
bacterial groups with validation references.

| Probe | | | |
|---|---|---|---|
| name | Sequence (5′ to 3′) | Target group | References |
| Non Eub | ACTCCTACGGGAGGCAGC | (Wallner et al., 1993) | (Wallner et al., |
| | | | 1993) |
| Eub338 | GCT GCC TCC CGT AGG AGT | Most bacteria | (Daims et al., |
| I | | | 1999) |
| Eub338 | GCA GCC ACC CGT AGG TGT | Planctomycetales | (Daims et al., |
| II | | | 1999) |
| Eub338 | GCT GCC ACC CGT AGG TGT | Verrucomicrobiales | (Daims et al., |
| III | | | 1999) |
| Bif164 | CAT CCG GCA TTA CCA CCC | Most Bifidobacterium spp. | (Langendijk et |
| | | and Parascardovia | al., 1995) |
| | | denticolens | |
| Lab158 | GGTATTAGCAYCTGTTTCCA | Most Lactobacillus, | (Harmsen et al., |
| | | Leuconostoc and Weissella | 1999) |
| | | spp.; Lactococcus lactis; all | |
| | | Vagococcus, Pediococcus | |
| | | and Paralactobacillus spp., | |
| | | Melisococcus, | |
| | | Tetragenococcus, | |
| | | Catellicoccus, Enterococcus | |
| Bac303 | CCA ATG TGG GGG ACC TT | Most Bacteroidaceae and | (Manz et al., |
| | | Prevotellaceae, some | 1996) |
| | | Porphyromonadaceae | |
| Erec482 | GCT TCT TAG TCA RGT ACCG | Most of the Clostridium | (Manz et al., |
| | | coccoides-Eubacterium | 1996) |
| | | rectale group (Clostridium | |
| | | clusters XIVa and XIVb) | |
| Chis150 | TTATGCGGTATTAATCTYCCTTT | Most of the Clostridium | (Franks et al., |
| | | histolyticum group | 1998) |
| | | (Clostridium clusters I and II) | |
| Rrec584 | TCA GAC TTG CCG YAC CGC | Roseburia subcluster | (Franks et al., |
| | | | 1998) |
| Prop853 | ATT GCG TTA ACT CCG GCAC | Clostridial cluster IX | (Walker et al., |
| | | | 2005) |
| Ato291 | GGT CGG TCT CTC AAC CC | Atopobium, Colinsella, | (Harmsen et al., |
| | | Olsenella and Eggerthella | 2000) |
| | | spp.; Cryptobacterium | |
| | | curtum; Mycoplasma | |
| | | equigenitalium and | |
| | | Mycoplasma elephantis | |
| Fprau655 | CGCCTACCTCTGCACTAC | Faecalibacterium prausnitzii | (Hold et al., |
| | | and related sequences | 2003) |
| DSV687 | TAC GGA TTT CAC TCC T | Most Desulfovibrionales and | (Devereux et al., |
| | | many Desulfuromonales | 1992) |

### Statistical Analysis

All statistical results were analysed in Prism 8 version 8.4.0 (GraphPad Prism 8, San Diego, California, USA). The FISH-FCM and organic acid production were analysed using two-way mixed ANOVA to compare different test substrates and time points. Where significant differences were found, a post hoc analysis was performed using Tukey multiple comparison tests. Statistical analysis was accepted at P<0.05 for all analyses.

### Results

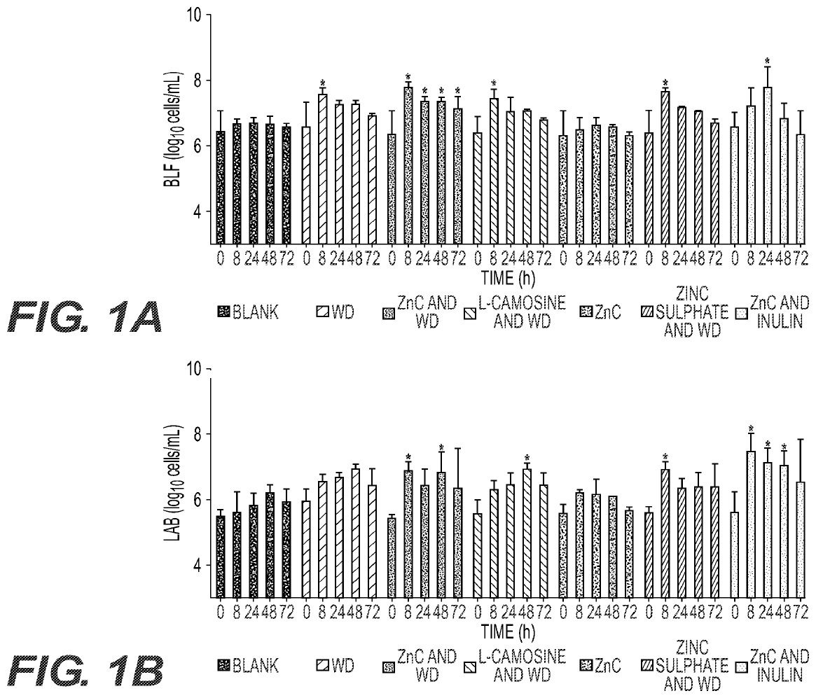

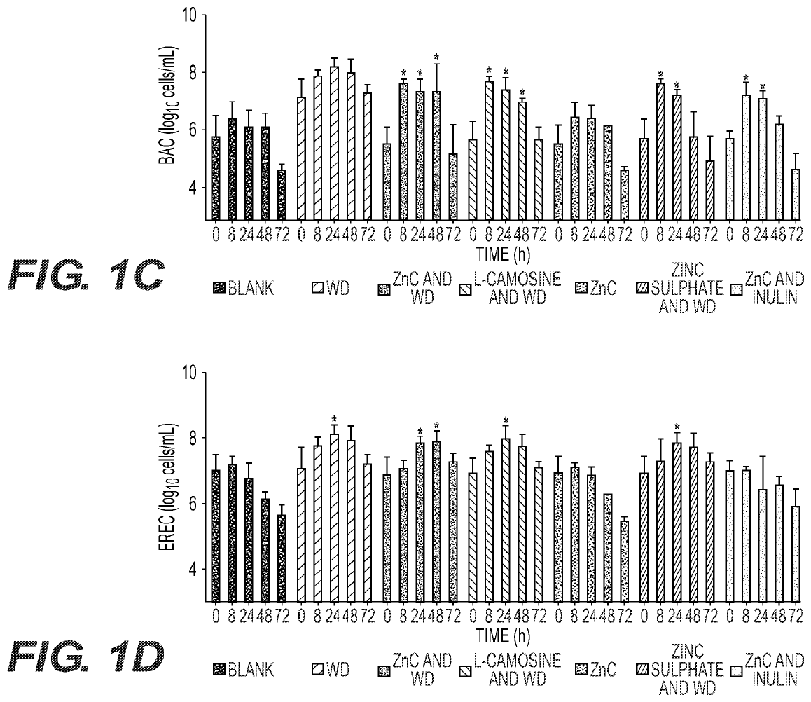

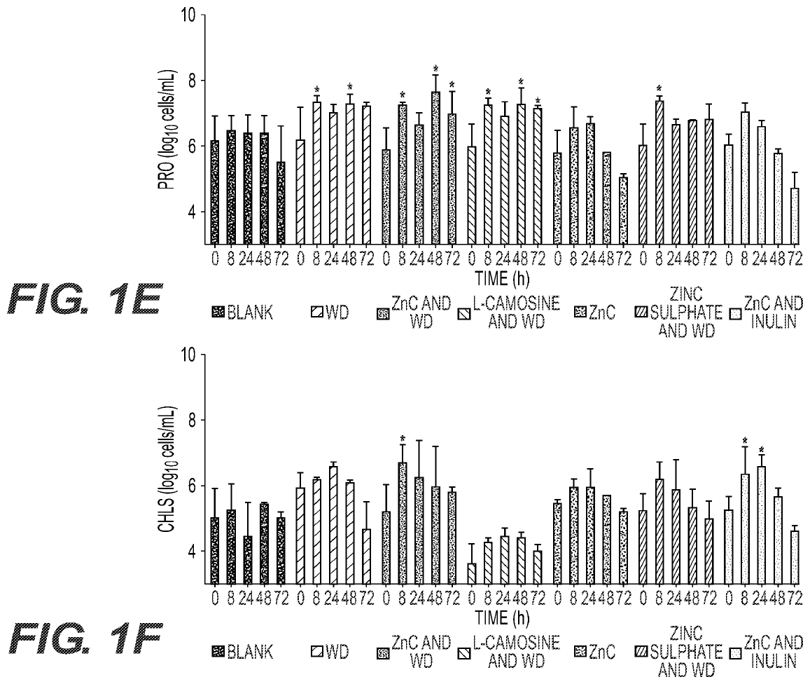

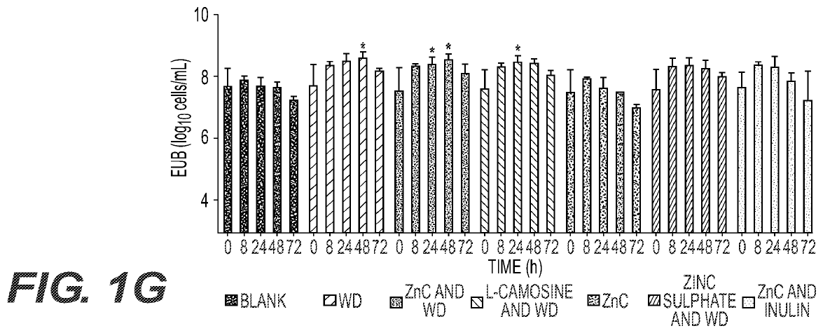

FIGS. 1A-1G illustrates bacterial enumeration (log₁₀ cells/mL) from in vitro batch culture after fermenting for 0, 8, 24, 48 and 72 h. The bacteria analyzed were Bifidobacterium spp. (Bif) (A), lactic acid bacteria (Lab) (B), Bacteroidaceae and Prevotellaceae (Bac) (C), Clostridial cluster XIVa and XIVb (Erec) (D), Clostridial cluster (Pro) (E) and Clostridium histolyticum group (Chis) (F) and total bacteria (Eub) (G). The vessels consisted of blank, wheat dextrin (WD), zinc carnosine (ZnC) and wheat dextrin (WD), L-carnosine and wheat dextrin (WD), zinc carnosine (ZnC), zinc sulfate and wheat dextrin (WD) and zinc carnosine (ZnC) and inulin vessels. Data is presented as mean±SD (n=3) and * shows significant difference (P<0.05) compared to the baseline (TO).

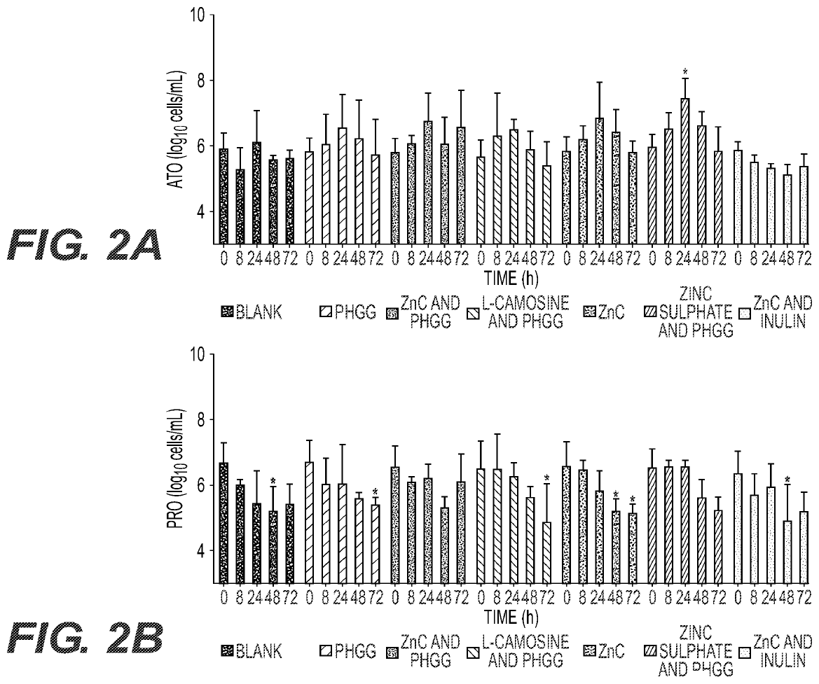

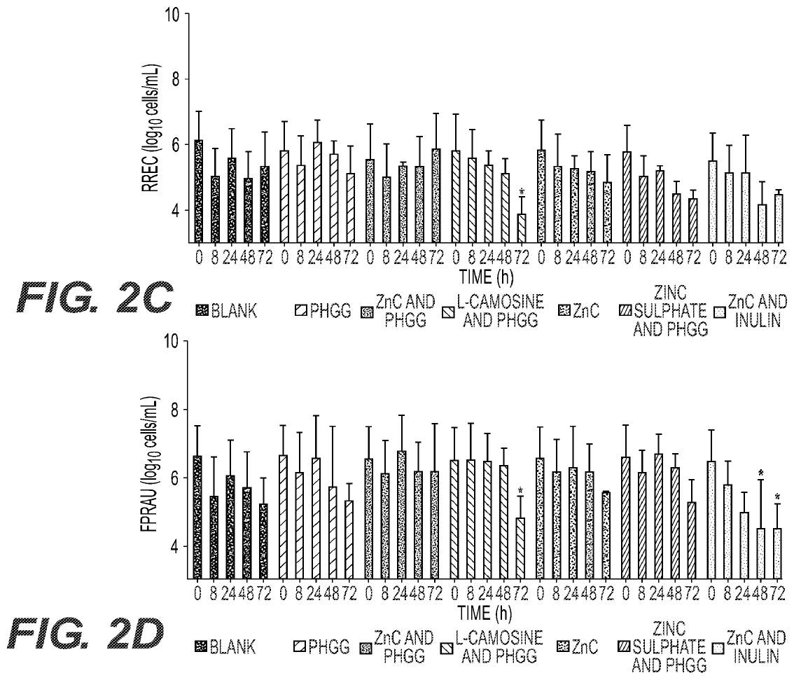

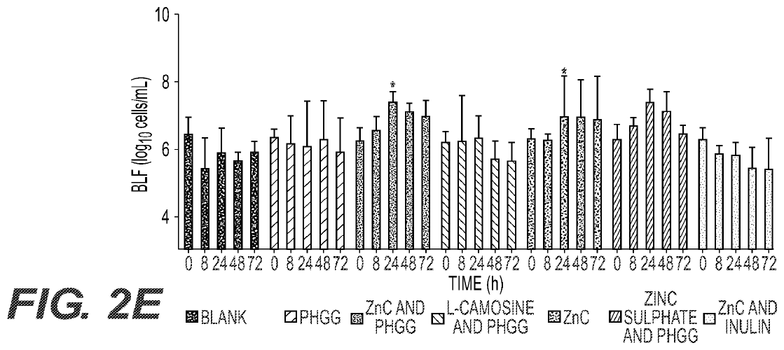

FIGS. 2A-2E illustrates bacterial enumeration (log 10 cells/mL) from in vitro batch culture after fermenting for 0, 8, 24, 48 and 72 h. The bacteria analyzed were Atopobium spp. (Ato) (A), Clostridial cluster (Pro) (B). Roseburia (Rrec) (C), Faecalibacterium prausnitzii (Fprau) (D) and Bifidobacterium spp., (Bif) (E). The vessels were blank, partially hydrolysed guar gum (PHGG), zinc carnosine (ZnC) and partially hydrolysed guar gum (PHGG), L-carnosine and partially hydrolysed guar gum (PHGG), zinc carnosine (ZnC), zinc sulfate and partially hydrolysed guar gum (PHGG) and zinc carnosine (ZnC) and inulin. Data is presented as mean±SD (n=3) and * shows significant difference (P<0.05) compared to the baseline (TO).

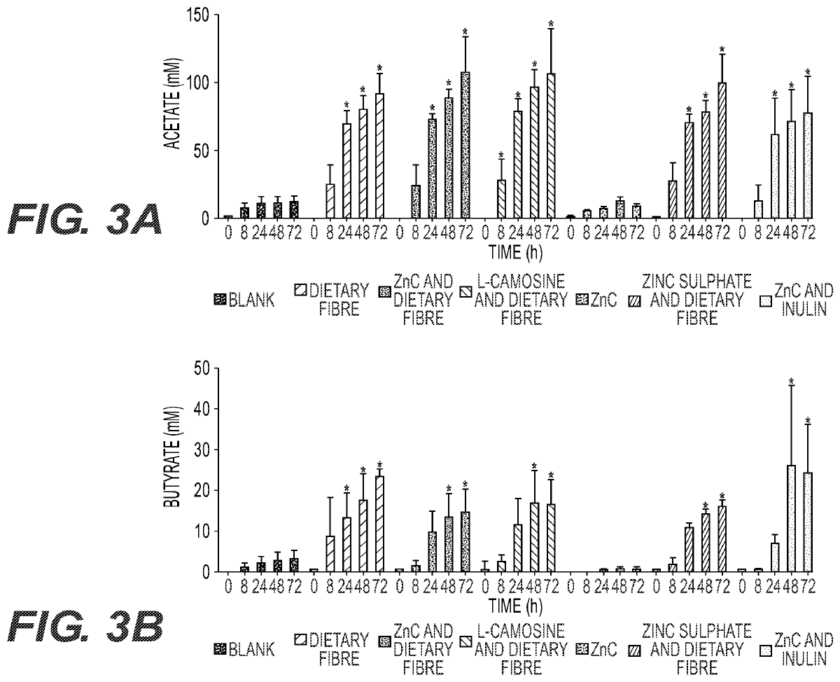

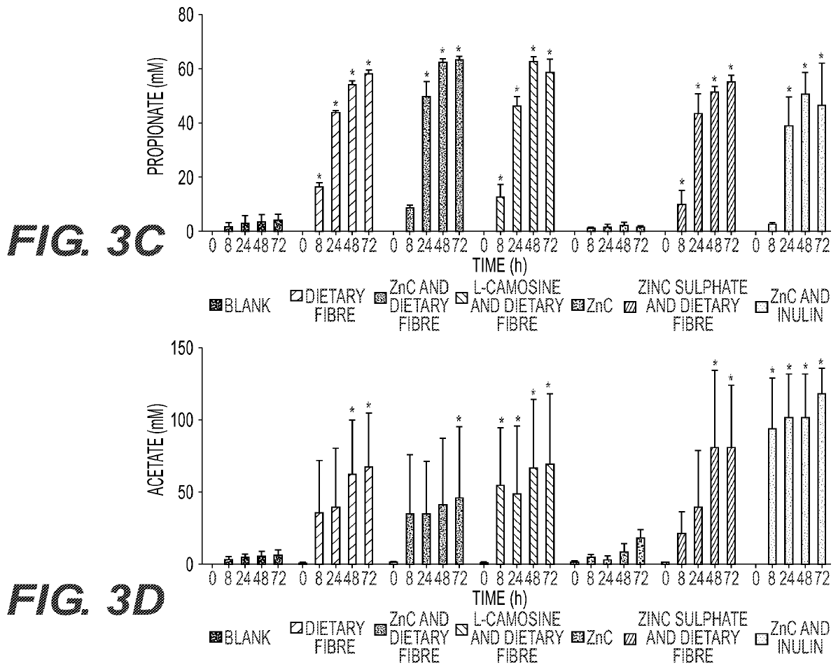

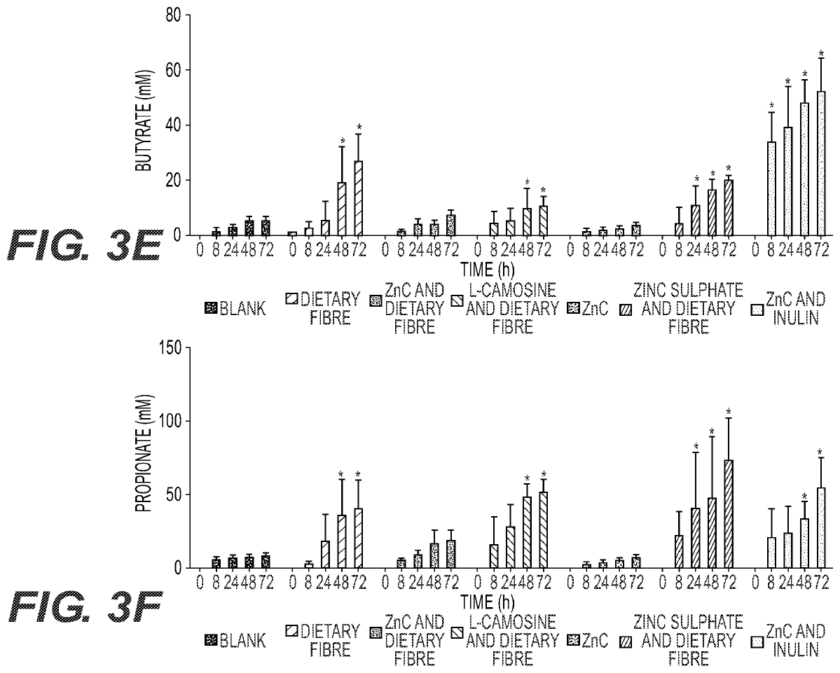

FIG. 3 illustrates organic acid production (mM) from in vitro batch culture fermentation. FIGS. 3A, 3B and 3C show (left hand side) the blank, wheat dextrin, zinc carnosine and wheat dextrin. L-carnosine and wheat dextrin, zinc carnosine, zinc sulfate and wheat dextrin and zinc camosine and inulin vessels. The FIGS. 3D, 3E and 3F consists of (right hand side) blank, partially hydrolysed guar gum, zinc camosine and partially hydrolysed guar gum, L-carnosine and partially hydrolysed guar gum, zinc carnosine, zinc sulfate and partially hydrolysed guar gum and zinc carnosine and inulin. All measures were taken at 0, 8, 24, 48 and 72 h. Organic acids measured were acetate, butyrate and propionate. Data are presented as mean±SD (n=3) and * shows significant difference (P≤0.05) compared to the baseline (TO).

Overall, the addition of ZnC to WD enhanced bacteria metabolism to a greater extent than in the PHGG. Total bacteria counts were elevated in all intervention test vessels, except ZnC vessel, until 48 h in the WD experiment as can be seen in FIG. 1G. There was a significant increase in total bacteria counts at 24 h in the WD and L-carnosine vessel (P=0.0402) with a 0.85 log 10 cells/mL increase. Similar 0.85-0.99 log 10 cells/mL increase was found at 48 h in the WD vessel (P=0.0410) and WD and ZnC vessel (P=0.0106), respectively. The WD vessel had significantly higher total bacterial counts compared to the blank and ZnC vessel at 48 h (P≤0.0356) and 72 h (P≤0.0410).

The WD vessel alone lead to a significant increase in Bifidobacterium spp, at 8 h compared to baseline (P=0.0124) whereas, the WD combined with ZnC resulted in a prolonged significant increase until the end of the experiment (P≤0.0473) (FIG. 1A). The WD and ZnC vessel increased the production of Bifidobacterium spp., by 0.5 log 10 cells/mL greater than that in the WD vessel alone. The significant increase in Bifidobacterium spp., was also shown when compared to the blank vessel (P=0.0063) and ZnC vessel (P=0.0070). Bifidobacterium spp. also significantly increased in the PHGG and ZnC vessels (P=0.0405) at 24 h compared to baseline with a 1.14 log 10 cells/mL (FIG. 2E). The inulin and ZnC vessel was significantly higher in Bifidobacterium spp . . . at 24 h compared to baseline (P=0.0009), the blank (P=0.0130) and ZnC vessel (P=0.0237).

Lactic acid bacteria was not found to be significant in the WD vessel (FIG. 1B). When WD was combined with either ZnC, zinc or L-carnosine a significant increase in lactic acid bacteria was observed. L-carnosine showed a significant increase in lactic acid bacteria early in the fermentation at 8 h increasing by 1.33 log 10 cells/mL whereas, vessels containing zinc alone did not significantly increase until 48 h but showed a similar growth (1.45 log 10 cells/mL). The inulin and ZnC vessel significantly increased lactic acid bacteria compared to baseline at 8, 24 and 48 h (P≤0.0018) and compared to blank at 8 and 24 h (P≤0.0242).

In the WD vessel alone no significant elevation in bacteroides was found but when combined with ZnC (P≤0.0007), zinc (P≤0.0070) and L-carnosine (P≤0.0206) a significant increase was present (FIG. 1C). The increase in bacteroides from baseline was greater in the WD and ZnC vessel (2.06 log 10 cells/mL) compared to the WD and L-carnosine (1.99 log 10 cells/mL) and WD and zinc (1.85 log 10 cells/mL) vessels.

Clostridium histolyticum group significantly increased in the WD and ZnC (P=0.0018) vessels by 1.5 log 10 cells/mL (FIG. 1F). There was also a significant increase (P=0.0059) in the inulin and ZnC vessel at 24 h with an increase of 1.36 log 10 cells/mL.

Clostridium coccoides-Eubacterium rectale group and Clostridial cluster IX significantly increased in the WD alone and in the WD combined with ZnC, zinc and L-carnosine (FIG. 1D and FIG. 1E).

Overall, the production of acetate, butyrate and propionate significantly increased throughout the duration of the fermentation (FIG. 3).

Acetate production significantly increased in all intervention test vessels, except ZnC vessel, at 24 h (P>0.0001), 48 h (P>0.0001) and 72 h (P>0.0001) compared to baseline in the WD experiment (FIG. 3A) The WD and ZnC vessel had the largest acetate production with an increase from baseline of 106.24 mM. In the PHGG experiment acetate was significantly elevated at 72 h compared to baseline in all other intervention test vessels, except for the ZnC vessel (FIG. 3D).

Butyrate production significantly increased in all of the intervention test vessels in both the WD and PHGG experiments, except for the ZnC alone vessel and the PHGG and ZnC vessel (FIGS. 3B and 3E). In both experiments the vessel containing inulin showed the greatest increase in butyrate; the PHGG and inulin vessel increased in butyrate production by 51.93 mM and the WD and inulin increased by 25.81 mM.

Propionate was significantly elevated at 24, 48 and 72 h in all of the intervention test vessels, except ZnC vessel, compared to baseline in the WD experiment (FIG. 3C). There was a similar increase in propionate vessels ranging from an increase of 46.46 to 63.33 mM in the WD experiment. In the PHGG experiment all intervention vessels increased in propionate levels at 48 and 72 h compared to baseline, except for the ZnC and PHGG and ZnC vessel (FIG. 3F).

### Discussion and Conclusion

Overall, the main findings of the study were that the ZnC supplementation combined with WD increased Bifidobacterium spp., throughout the duration of the study but this did not occur in the L-carnosine and zinc vessels. Vessels containing WD showed a greater alteration in bacterial metabolism compared to the PHGG vessels.

Regulation of zinc is important as it can be toxic to bacteria if present in excess concentrations. On the other hand, zinc deficiency has been associated with infectious diseases and IBD; therefore, zinc may be essential for the immune system and preventing inflammation.

Zinc has many functions one of which is to help maintain intestinal barrier integrity; alteration in gut barrier function allows permeation of detrimental microorganisms, antigens and proinflammatory factors. In a study using Caco-2 cells, depletion of zinc led to proteolysis of occludin which is a key tight junction protein. When intake of ZnC was consumed, intestinal barrier function was maintained or improved in healthy volunteers under high stress or when diarrhoea was induced. Improvements in barrier function may be important for diseases like IBD which have previously been shown to improve in terms of UC clinical outcomes and remission following ZnC supplementation (150 mg/day for 1 week).

This study is novel and the first to investigate the effects of ZnC supplementation on the human gut microbiota. In the few studies which are available that have investigated the impacts of zinc, the gut microbiota has been assessed mainly by measuring the growth of pathogenic bacteria in pure culture. For example, when bacterial infection and inflammation occur there is an elevation in zine concentrations. Campylobacter and Escherichia coli have metalloregulatory proteins, i.e. zinc uptake regulator (Zur), which regulates gene expression of znuABC dimming the intake of zinc, therefore showing survival in zinc deprived environments.

Some lactic acid bacteria, especially Lactobacillus acidophilus WC 0203, have been found to uptake zinc. In the current study, Bifidobacterium spp., were found to be significantly increased in the vessels containing WD and ZnC and PHGG at various times compared to baseline. The WD and ZnC vessel showed a significant increase in Bifidobacterium spp., until the end of the experiment. Lactobacillus spp. was also significantly elevated in the WD and ZnC vessels compared to baseline. The WD and Znc Bifidobacterium spp., and Lactobacillus spp. are considered as health-positive bacteria as shown by reducing acute diarrhoea and improve immunity in elderly participants along with other health outcomes such as decreasing inflammatory status.

Plant-based foods such as nuts, grains and pulses contain phytate. Phytate binds strongly to zinc ions inhibiting absorption leading to an increase in excretion. When zinc is combined with amino acids such as histidine it increases zinc bioavailability. Absorption by bacteria is dependent on pH, temperature and microorganism species.

However, there was a significant increase in this study, with a large standard deviation present, in the Clostridium histolyticum group (Clostridium cluster I and II) in the WD and ZnC vessel and inulin and ZnC vessel. Clostridium histolyticum includes some pathogenic bacteria that can produce endotoxins and has previously been associated with disease states such as UC. The bacterial group can produce collagenase and proteinases which degrades tissues.

L-carnosine, found in skeletal muscle, kidney and the brain, is a good carrier for zinc and has been shown to play a role in wound healing but may also be important to improve neurodegenerative and neurological disorders. Cerebral ischemia, which leads to severe brain damage, may be improved by camosine in early stage recovery as an increase in expression of glutamate via EAACl, leads to GABA synthesis, increasing extracellular GABA as well as increasing recovery in mitochondrial energy metabolism. Research is emerging assessing the link between the gut and brain, named the gut-brain axis, and the supplementation of ZnC may be an interesting concept to investigate to gain understanding into whether ingesting ZnC could lead to alterations in neurotransmitters.

Previous research has shown an association between zinc intake and depression. A randomised, double-blind, placebo-control found, in 44 participants with major depression, that combining zinc supplementation (25 mg per day for 12 weeks) with antidepressants may help to reduce severity of symptoms. This finding is supported by a recent Japanese epidemiological study showing around an inverse association between zinc intake and anxiety and depression in about 2000 participants. Supplementation of ZnC may be a novel treatment to target gut barrier function as well as neural aspects associated with GI disorders such as Irritable Bowel Syndrome (IBS).

Additionally, adding probiotic strains may diminish pathogenic bacteria. Interestingly, components of ZnC separately have been found to help neurodegenerative, neurological and psychiatric disorders therefore further research investigating the gut-brain axis could be of interest.

What is claimed is:

1. A nutritional supplement composition comprising: a dietary fiber and zinc carnosine.

2. The nutritional supplement composition according to claim 1, wherein the amount of wheat dextrin is between about 2600 mg and about 15,000 mg per serving.

3. The nutritional supplement composition according to claim 1, wherein the composition comprises about 10 mg to about 500 mg of zinc carnosine per serving.

4. The nutritional supplement composition according to claim 1, wherein the dietary fiber is wheat dextrin, hydrolyzed guar gum, inulin, or a combination thereof.

5. The nutritional supplement composition according to claim 4, wherein the dietary fiber has a degree of polymerisation (DP) of 12-25.

6. The nutritional supplement composition according to claim 4, wherein the dietary fiber is wheat dextrin.

7. The nutritional supplement composition according to claim 6, wherein the amount of wheat dextrin is between about 2600 mg and about 7400 mg per serving.

8. The nutritional supplement composition according to claim 1, wherein the composition includes one or more polyvalent metals, vitamins, minerals, enzymes, microorganisms, botanicals, or a combination thereof.

9. A method of improving gut metabolism comprising administering the nutritional supplement composition according to claim 1.

10. A nutritional supplement composition according to claim 1 for use in enhancing gut health in a subject.

11. A nutritional supplement composition according to claim 1 for use in enhancing gut health in a subject by increasing the number of bacteria of Bifidobacterium spp. in the gastrointestinal tract or feces of the subject.

12. The nutritional supplement composition according to claim 11, wherein the number of bacteria of Bifidobacterium spp. in the gastrointestinal tract or feces of the subject is increased in comparison to the number of bacteria of Bifidobacterium spp. in the gastrointestinal tract or feces of the subject prior to administration of the nutritional supplement composition.
